(19) United States
(12) Patent Application Publication
(10) Pub. No.: US 2026/0369400 A1
(43) Pub. Date: Nov. 19, 2026

(54) INTEGRATED AED INCIDENT REPORTS

(71) Applicant: Avive Solutions, Inc., Brisbane, CA (US)

(72) Inventor: Rory M. BEYER, San Mateo, CA (US)

(21) Appl. No.: 19/676,580

(22) Filed: May 13, 2026

Related U.S. Application Data
(60) Provisional application No. 63/805,430, filed on May 14, 2025.

(51) Int. Cl. G16H 40/67; G16H 15/00

(52) U.S. Cl. CPC G16H 40/67; G16H 15/00

(57) ABSTRACT

Systems and methods are disclosed for generating enhanced incident reports associated with the emergency use of AEDs. The incident reports integrate AED-generated data with patient-related data obtained from one or more independent health monitors. These monitors may include wearable or implantable medical devices, consumer-grade health trackers, and other devices capable of detecting pre-incident physiological signals. Data from the AED and from one or more associated monitors are collected, merged, and incorporated into a comprehensive incident report. The report may be made accessible via a medical records system, incident server, or AED management platform to assist emergency responders and medical personnel in evaluating the patient’s condition before, during, and after the AED deployment. The integrated report provides a more complete temporal context for the cardiac event and improves clinical decision-making during time-critical interventions.

### CROSS-REFERENCE TO RELATED APPLICATIONS

This application claims priority of U.S. Provisional Application No. 63/805,430, filed on May 14, 2025, which is incorporated by reference herein in its entirety.

### FIELD

The present disclosure relates generally to automated external defibrillators (AEDs) and AED incident reports that document emergency uses of an AED and provide incident related information detected by other monitors that are independent of the AED.

### BACKGROUND

Automated external defibrillators (AEDs) are portable, life-saving tools designed to automatically check for life-threatening heart rhythms associated with sudden cardiac arrest and send an electrical shock to the heart to try to restore a normal rhythm when shockable heart rhythms are detected. AEDs are commonly deployed in public and private environments to enable timely response to cardiac emergencies. In addition to delivering life-saving therapy, some modern AED systems integrate, or interface with, platforms that provide enhanced features such as remote connectivity, location awareness, alert delivery, user-facing displays, and device monitoring.

Designing a platform suitable for such applications involves selecting an appropriate execution environment, service framework, and system architecture capable of supporting the desired functionality. This selection typically involves balancing competing considerations related to computational capability, responsiveness, power consumption, communication reliability, and long-term operability in power-constrained environments.

Platforms that emphasize richer functionality such as higher-bandwidth communications, secure bidirectional data exchange, graphical user interfaces, and support for complex tasks can provide flexibility and extensibility. However, such platforms also typically involve increased startup latency, greater energy consumption, and longer active operating periods, which can be undesirable in devices expected to remain in low-power standby states for extended durations while still responding promptly to triggering events. Conversely, platforms optimized for low power consumption and rapid responsiveness may be well suited for handling time-sensitive operations and basic device coordination tasks. However, such platforms are typically limited in their ability to support more advanced features and services. These tradeoffs are particularly pronounced in emergency medical device systems such as AEDs, which may be required to conserve battery power over months or years of standby operation while providing periodic status information to remotely located servers, yet still provide immediate responsiveness when activated.

Accordingly, there exists a continuing need for improved execution environments, service platforms, and system architectures used in emergency medical device applications.

### SUMMARY

A variety of AEDs, monitors and methods for generating integrated incident reports are described. In one method aspect, a server infrastructure receives incident data from a first AED regarding a use of the first AED in connection with a medical incident. The server infrastructure also receives or retrieves a monitor identifier that identifies a first monitor that is independent of the first AED wherein the first monitor (i) is/was in a vicinity of the medical incident during the use of the first AED, (ii) was pre-associated with the first AED prior to the medical incident, (iii) is/was able to detect the first AED during use of the first AED, or (iv) was associated by identifying a patient following the use of the first AED. Monitor data is requested from a monitor management server associated with the first monitor using the monitor identifier and the server infrastructure receives the requested monitor data. The server infrastructure generates an incident report that combines at least some of the received AED incident data and at least some of the received monitor data.

In some embodiments, the first monitor was pre-associated with the first AED prior to the medical incident and the monitor identifier is retrieved from an AED management database that stores information about a multiplicity of AEDs, including the first AED.

In some embodiments, the monitor identifier is received from the first AED. In some embodiments, during use of the first AED in connection with the medical incident, the first AED scans for nearby monitors and the monitor identifier is obtained by the first AED as part of the scanning for nearby monitors. In some embodiments, the scan is performed using one of a near field scanning protocol; an RFID scan; a Bluetooth scan; a Bluetooth Low Energy scan; a Wi-Fi scan; a Zigbee scan or a Z-Wave scan.

In some embodiments, the monitor identifier was input into the first AED after the use of the first AED in connection with the medical incident.

In some embodiments, the monitor identifier is received from the monitor management server during or after the use of the first AED in connection with the medical incident.

In some embodiments, an incident record is created in response to reception of incident data from the first AED, and the incident report is generated based at least in part on information in the incident record. The incident report may then be made available to a requestor.

In some embodiments, the incident report is made available to a requestor during at least one of: (i) the use of the first AED; and (ii) the medical incident.

In another aspect a novel AED is described. The AED includes (i) a shock discharge capacitor; (ii) at least one defibrillation electrode pad, the shock discharge capacitor and the at least one defibrillation electrode pad being suitable for delivering a defibrillation shock to a potential cardiac arrest incident victim during a first emergency use of the AED; and (iii) one or more processors. The processor(s) is/are collectively configured to cause the AED to electronically scan for nearby monitor devices in association with the first emergency use of the AED. The processor(s) also cause a detected device list to be electronically transmitted directly or indirectly to a remotely located server infrastructure. The detected device list identifies at least one device detected by the electronic scan. The processor(s) also generates a first incident log that contains event information related to the first emergency use of the AED and causes the first incident log to be electronically transmitted directly or indirectly to the remotely located server infrastructure. The first incident log includes at least one of a first incident identifier, and elements that can be used to generate the first incident identifier. The server infrastructure is configured to receive incident logs from a multiplicity of different AEDs.

In some embodiments, the AED further includes an RFID reader that, at least in part, performs the scan for nearby monitor devices. In some embodiments, the AED further includes a BLE module that, at least in part, performs the scan for nearby monitor devices.

In some embodiments, the AED further comprises a display screen. And the processor(s) is/are further configured to cause the incident identifier to be rendered on the display screen after the first emergency use of the AED.

In some embodiments, the processor(s) is/are further configured to cause status messages to be periodically sent to the server infrastructure during the first emergency use of the first AED.

In another aspect an AED includes: (i) a shock discharge capacitor, (ii) at least one defibrillation electrode pad, the shock discharge capacitor and the at least one defibrillation electrode pad being suitable for delivering a defibrillation shock to a potential cardiac arrest incident victim during a first emergency use of the AED, (iii) a display screen, and (iv) one or more processors. The one or more processors are collectively configured to, in association with the first emergency use of the first AED, generate a first incident log that contains event information related to the first emergency use of the first AED. The processor(s) also cause the first incident log to be electronically transmitted directly or indirectly to a remotely located server infrastructure. The first incident log includes at least one of a first incident identifier, and elements that can be used to generate the first incident identifier. The server infrastructure is configured to receive incident logs from a multiplicity of different AEDs. The processor(s) is/are also configured to, cause the AED to receive a user input that provides at least one of (a) a monitor identifier that identifies a monitor associated with a patient that the first AED was used on during the first emergency use of the first AED, or (b) a patient identifier that identifies the patient. The received monitor identifier or patient identifier is forwarded to the server infrastructure.

In another aspect, a patient monitoring system is disclosed. The patient monitoring system includes a monitor server infrastructure and a first monitor located remotely relative to the monitor server infrastructure. The first monitor is configured to (i) monitor a physiological characteristic of a patient, (ii) send monitor data regarding the monitored patient’s physiological characteristic to the monitor server infrastructure, (iii) detect a deployment of a first defibrillator, and (iv) notify the monitor server infrastructure of the detection of the deployment of the defibrillator. The monitor server infrastructure forwards at least some of the received monitor data to a remotely located AED incident management server infrastructure to facilitate inclusion of at least some of the forwarded monitor data in an incident report that combines incident data collected by the first defibrillator during the deployment of the first defibrillator. In some embodiments, the first defibrillator is an automated external defibrillator (AED).

In some embodiments, in response to being notified of the detection of the deployment of the first defibrillator, the monitor server infrastructure sends an incident query to the AED incident management server infrastructure. At least some of the forwarded monitor data is sent to the AED incident management server infrastructure in response to a monitor data request sent by the AED incident management server infrastructure to the monitor server infrastructure.

### BRIEF DESCRIPTION OF THE DRAWINGS

The invention and the advantages thereof, may best be understood by reference to the following description taken in conjunction with the accompanying drawings in which:

FIG. 1 is a block diagram diagrammatically illustrating a system in accordance with an embodiment.

FIGS. 2A-2C are flow charts illustrating a method of generating an AED incident report that includes pre-incident data obtained from monitors that are independent of the AED in accordance with an embodiment, from the perspective of the AED, the incident report server and the monitor management server respectively.

FIG. 3 is a flow chart illustrating a method of generating an AED incident report that includes pre-incident data obtained from monitors that are independent of the AED in accordance with another embodiment.

FIG. 4 is a flow chart illustrating a method of associating AED incident data with monitor data by way of a management platform.

FIG. 5 is a flow chart illustrating a method of associating AED incident data with monitor data by way of a user interface on the AED or an AED app.

FIG. 6 illustrates a sample incident report that integrates data from an independent monitor with AED data.

FIG. 7 is a block diagram illustrating electrical components of a representative defibrillator unit.

FIG. 8 is a block diagram illustrating electrical components of a representative interface unit.

In the drawings, like reference numerals are sometimes used to designate like structural elements. It should also be appreciated that the depictions in the figures are diagrammatic and not to scale.

### DETAILED DESCRIPTION

The present disclosure relates generally to incident reports created in connection with the use of defibrillators such as automated external defibrillators.

As described in the background, AED incident reports can be very helpful to medical practitioners because it gives the medical practitioner some visibility as to what happened during a cardiac arrest. A limitation of existing reports is that at best, they begin monitoring the patient at the point in time when the AED is deployed, which is after the patient has gone into cardiac arrest. With the proliferation of electronic devices that monitor patients, there are situations in which a cardiac arrest victim may have been wearing or otherwise be under the view of one or more monitors that have information that would also be helpful to medical personnel. This could include formal medical devices such as implantable cardiac monitors (ICM), wearable cardiac monitors (e.g., a Holter monitor), portable ECG/EKG devices, ECG patches, glucose monitors, pulse oximeters (SpO₂ monitors), photoplethysmography (PPG) monitors or a variety of other medical devices. Alternatively, or additionally, it could include a less formal device such as a smart watch, a fitness tracker, a heart rate monitor, other “smart” device such as rings, armbands or earbuds, wearable PPG monitors, heart rate monitors, heart rate variability (HRV) monitors, single lead ECGs, sleep staging and stress/recovery monitors (which are currently features of many wearable devices), etc.

Some monitors, such as implantable and wearable cardiac monitors, have systems in place for their data to be sent to the patient’s cardiologist. Unfortunately, even when the victim is wearing or otherwise under the view of such a monitor, the relevant monitor data is very rarely available to emergency medical personnel who may be attending to the victim very shortly after a cardiac arrest incident occurs. Cardiac arrest related incident data from more consumer-oriented monitors is rarely made available to any attending medical personnel. The present disclosure describes techniques and devices for integrating independent monitor data into AED usage incident reports in a manner that can give medical personnel better visibility of a patient’s condition before, during, and immediately after a potential cardiac arrest incident.

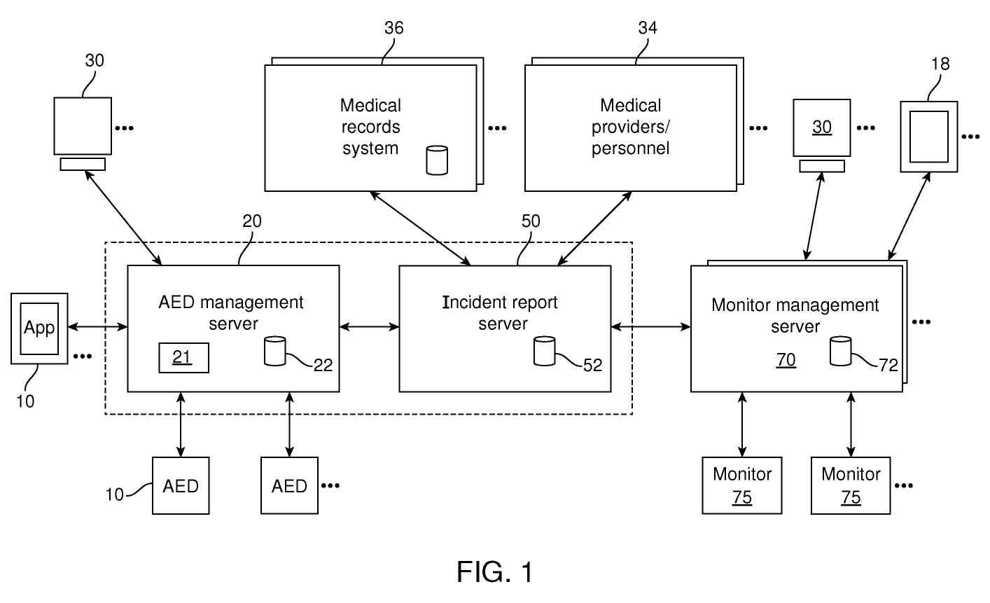

FIG. 1 illustrates a representative system in accordance with an embodiment. The system includes an AED management server infrastructure 20 in communication with a multiplicity of connected AEDs 10, an incident report server infrastructure 50, and a monitor management server infrastructure 70. The AED management server infrastructure 20 communicates directly or indirectly with a multiplicity of managed AEDs 10. In some embodiments, the AED management server infrastructure (referred to as AED management server 20 herein) may host an AED management platform 21 that may be accessed by a variety of computing devices 18, 30 etc. (sometimes referred to as administrator computing devices) that AED owners, administrators and other interested parties can utilize to manage their AEDs. In some circumstances a mobile device 18 may include a defibrillator app that facilitates communications with the AED management server 20. The AED management server typically includes an AED management database 22 that stores relevant information about the managed AEDs.

The incident report server infrastructure (referred to as an incident report server 50 herein for simplicity) is arranged to create and/or make AED incident reports available to interested parties. The incident report server 50 may be accessed by medical records systems 36 (such as electronic patient care record (EPCR) systems), medical providers/personnel/facilities 34 (e.g., doctors, emergency medical technicians, hospitals, etc.), and other interested parties in order to obtain incident reports that provide relevant medical and other information about emergency usages of an AED. The incident report server 50 includes an incident database 52 that contains data useful in creating incident reports and/or the incident reports themselves.

In some embodiments, some or all of the functionalities of the incident report server 50 are integrated as a part of AED management server 20, whereas in others it may be a separate system or part of a different system. The potential integration is emphasized by the dashed lines surrounding the two systems in FIG. 1. In other embodiments, the incident report server infrastructure may be alternatively or additionally integrated with an EPCR system. In still other embodiments, updates received by the incident report server infrastructure may be automatically pushed to relevant EPCR systems.

The monitor management server infrastructure (referred to as monitor management server 70 herein for simplicity) communicates directly or indirectly with a multiplicity of managed monitors 75. Like the AED management server, the monitor management server 70 can typically be accessed by a variety of administrator computing devices 18, 30 etc.

Health monitors or monitoring systems 75 typically include one or more biometric sensors that detect physiological data – such as ECG, heart rhythm, pulse, heart rate variability, blood flow, blood volume, glucose levels, oxygen saturation, sleep staging, stress/recovery, etc. – and an interface for communicating this information to the user or a remote system. In some cases, all monitoring functionality is integrated into a single unit. In other configurations, the monitor includes a base unit that is physically separate from the sensor. This base unit, located near the sensor, often provides a user interface that supports features such as alerts, user controls, real-time feedback, data visualization, and interaction with the collected data. The base unit may also handle communication with a remotely located monitoring or management server. In some configurations, a mobile application running on a smartphone or tablet acts as the base unit, receiving data from the sensor and serving as the interface for both the user and any connected health management platforms. As used herein, unless the context dictates otherwise, the term monitor is intended to refer to the monitoring system, which may include any of these configurations.

The Applicant has developed connected AEDs that are well suited for use as AEDs 10 in the illustrated system – although other AEDs may be used as well. By way of example, suitable AEDs are described in U.S. Patent Nos. 10,773,091 (P006E) and 11,452,881 (P016A), which are incorporated by reference. Applicant has also described systems and techniques for making AED incident reports available to medical personnel and other interested parties. By way of example, some systems and processes that make it much easier for medical providers/personnel 34 and medical record system 36 to access AED incident reports are described in U.S. Patent Application No. 17/217,738 (P022), which is incorporated herein by reference. Sample AED incident reports are illustrated in U.S. Patent Publication Nos. 2024-0366953 (P027A) and 2024-0371511 (P027B), each of which is incorporated herein by reference.

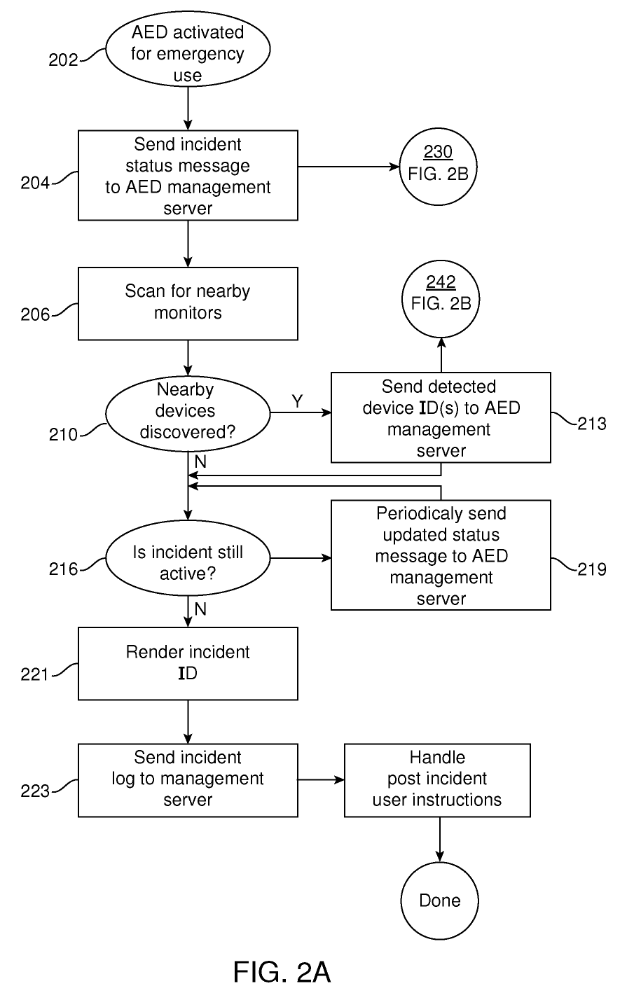

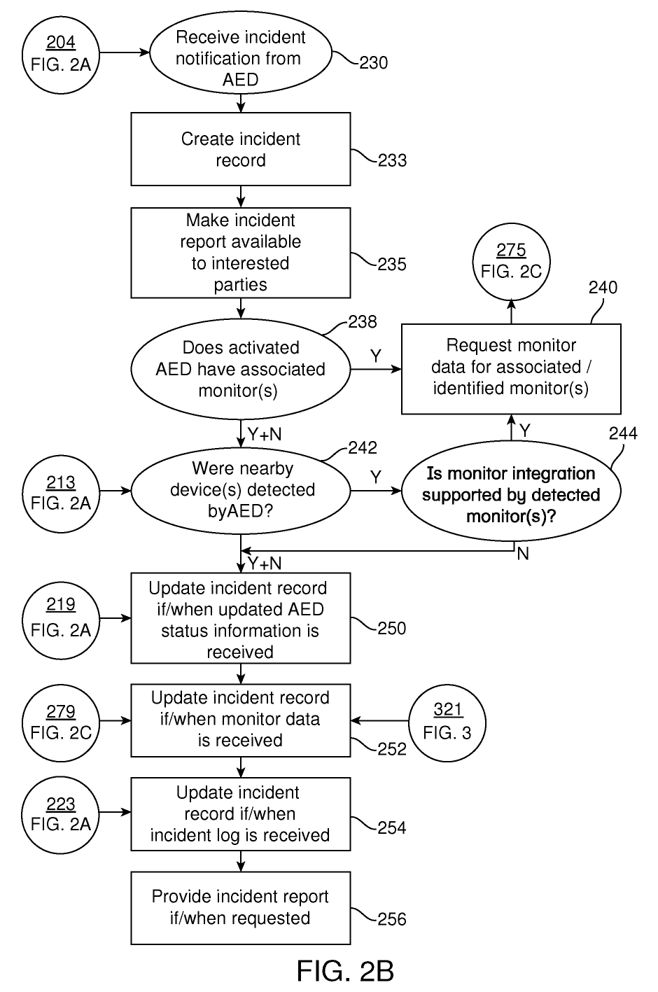

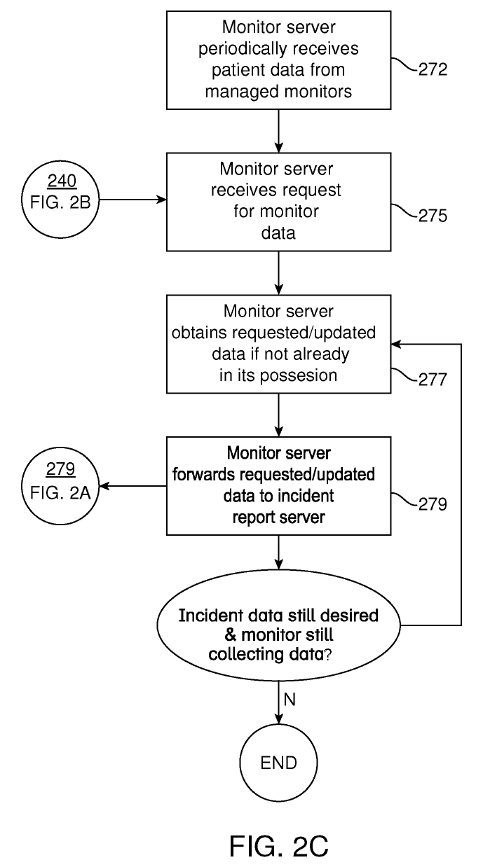

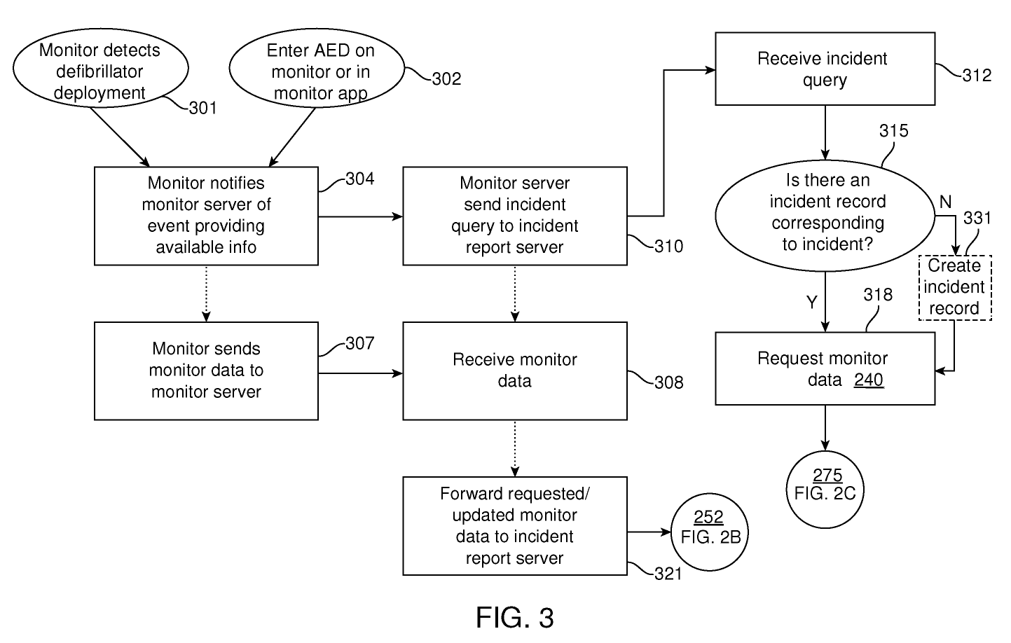

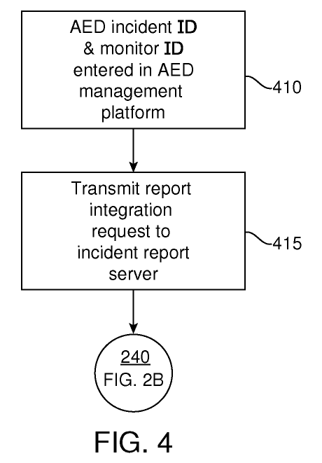

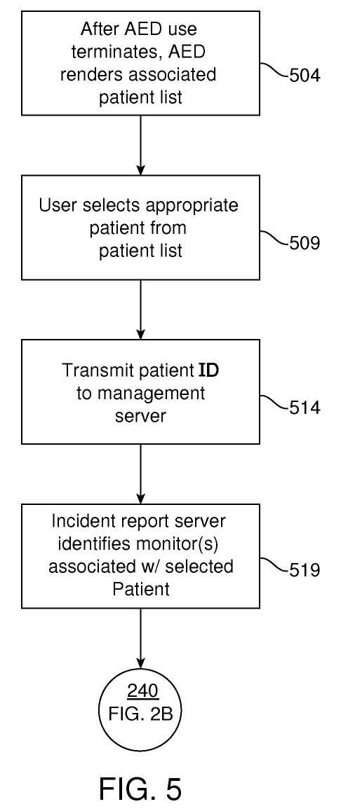

As outlined above, there are significant advantages to providing AED incident reports that supplement information provided by an AED with information provided by independent monitors to give medical personnel and other interested parties a more comprehensive overview of a potential cardiac arrest incident than can be obtained by information provided by the AED alone. Referring next to FIGS. 2A-2C, one approach to creating AED incident reports that include pre-incident data obtained from independent monitors in accordance with this disclosure will be described.

FIG. 2A illustrates a representative process from the perspective of an AED. The method begins when a selected AED 10 is activated for emergency use. Block 202. When the AED is activated, a unique incident ID is implicitly or explicitly assigned to the incident/session. The nature of the incident ID may vary widely, but by way of example, in some implementations it may be as simple as a concatenation of the AED’s serial number and a timestamp of the time that the AED was powered on, or a hash thereof. The AED then sends a status message (sometimes referred to as an incident status message) to the AED management server 20. Block 204. The contents of the status message may vary widely, but in a described embodiment has fields including the serial number or other unique identifier for the AED, the AED’s power on timestamp, an incident ID (to the extent it isn’t defined by the previous two fields), the geolocation of the AED and other relevant information. Although not required, preferably the AED periodically sends updated status messages to the management server throughout the incident as described below so that the management server has good real-time visibility as to what is happening with this deployment of the AED. A few suitable incident status messages and AEDs configured to send such status messages are described in U.S. Patent Nos. 10,737,105 (P006A); 10,773,091 (P006E); 11,452,881(P016A); 11,077,312 (P016B) and 11,640,755 (P021X1), each of which are incorporated herein by reference.

In some embodiments, the AED is configured to scan for nearby monitors. Block 206. The scanning can be performed in accordance with any suitable near field scanning protocol(s), as for Bluetooth or Bluetooth Low Energy (BLE) protocols, Wi-Fi protocols, RFID protocols, Zigbee or Z-Wave protocols, etc. If one or more nearby monitors are discovered (block 210), the ID(s) of the detected monitor(s) is sent to the AED management server. Block 213. To facilitate explanation, we discuss scanning for nearby monitors. However, in practice, the AED will often simply scan for nearby devices using the underlying protocol without making any judgements as to whether those devices are monitors of interest. Rather, the list of detected devices is simply forwarded to the management server which makes the determination of whether any monitors of interest were detected. Since there are many devices in existence that utilize the described protocols, it should be appreciated that statistically, many of the detected devices will not be monitors of interest.

As long as the AED remains in active use (Block 216), it periodically sends updated incident status messages to the management server. Block 219. Such incident status messages may contain a variety of information about the incident including information such as the current instruction state of the AED, the time of any cardiac rhythm analyses performed by the AED, as well as the corresponding heart rhythm classifications and/or shock/no-shock decisions, the time and nature of any defibrillation shocks delivered by the AED, etc. By way of example, some of the patents referenced above describe contents of a few representative status messages. This in-incident data can be used by the recipient servers to create real time incident reports based on data provided by the AED. Some representative incident report generating systems that support both real-time and post incident reporting are described in co-owned U.S. Patent Application No. 17/217,738 (P022), and U.S. Patent Publication Nos. 2024-0366953 (P027A) and 2024-0371511 (P027B), each of which are incorporated herein by reference.

When an AED is deployed, its pads will often be on the patient for several minutes before professional emergency services (e.g., EMS) arrive. When EMS arrives, they will typically take the AED’s electrode pads off of the patient and apply their own cardiac monitor/defibrillator or advanced cardiac life support (ACLS) device to the patient. When the AED’s electrode pads are removed, or the AED is powered off, the AED’s use is completed. (Reflected by the “No” branch from block 216).

In some embodiments, the AED will render the incident ID and/or a bar code that embeds the incident ID when the use of the AED has been completed. Block 221. When the incident ID is rendered apart from a bar code, it may take the form of an alphanumeric string or any other suitable form. When a bar code is rendered, the bar code may also embed a URL of a website from which an incident report can be obtained. Thus, the incident ID or the bar code may be used by medical personnel and/or other appropriate parties to access the incident report if/when desired.

In parallel, the AED sends an incident log file that was generated during use of the AED to the management server. Block 223. Relevant information from the log file is used by the incident report server to add additional information to the incident record, and any requested incident reports. For example, in some embodiments, the log file includes the patient’s ECG as detected by the AED together with timestamps showing when significant events occurred, as for example, when the AED was turned on, when the pads were placed, when different events including heart rhythm classification and shock delivery occurred, etc. Some representative processes for displaying and utilizing incident identifiers, as well as some representative log file generation and use scenarios that simplify the process of getting incident reports into the hands of medical personnel who can benefit most from such incident reports are described in the incorporated U.S. Patent Application No. 17/217,738 (P022).

In some situations, after the log file has been forwarded to the management server, the AED’s job is effectively done. However, in some situations, a user with the AED in hand may utilize the AED’s user interface to perform various tasks that can be helpful in either associating a monitor with the incident, or facilitating access to the incident report. Block 225. For example, in some embodiments, a family member, friend or other user with knowledge of a monitor associated with the victim can input the monitor’s ID via the AED’s user interface. The AED then sends the monitor ID to the management server either automatically or upon instruction from the user. This allows the management server to make an association between the designated monitor and the incident. In some embodiments, the AED’s user interface includes a dropdown menu (not shown) or other GUI widget that provides a list of supported monitors (i.e., a list of monitor types that the system can integrate data from). For example, the list may include X ECG patch, Y smart watch, Z smart ring etc. In some embodiments, selection of the monitor type prompts the rendering of a dialog box that allows the user to enter a specific monitor ID. In some embodiments, identification of monitor type additionally or alternatively prompts the AED to query for that type of monitor to obtain the monitor ID.

In some implementations, the monitor is configured to detect that an AED is in use in the vicinity. Applicant has described AEDs that broadcast status messages during emergency use in U.S. Patent Nos. 11,452,881 and 11,839,770, each of which is incorporated by reference. Such status messages may include information such as an incident ID, and have sufficient information such that the monitor can determine that an emergency use of the AED is occurring. The broadcasts may take the form of BLE advertisements or may utilize other suitable wireless formats. When the monitor receives a broadcast that indicates that a nearby AED is in use, it generates a prompt such as “do you want to share recent data with this emergency”. When the monitor has a screen, this prompt can be displayed on the monitor’s screen. When the monitor has a speaker, the prompt may be an audible prompt. Of course, the actual nature of the prompt will depend on the nature and capabilities of the particular monitor. If/when, a user indicates that they would like to share its data, the monitor informs the monitor management server 70. In turn, the monitor management server contacts the AED management server using an API call that effectively tells the AED management server to associate the monitor data with the accompanying AED incident ID.

In another example described in more detail below with respect to FIG. 5, when the AED is associated with one or more individuals (e.g., a patient to which the AED was prescribed, patients at a caregiving facility, family members for a home AED, etc.), the AED can be configured to render a patient list after the AED use terminates. A user may then identify the individual/patient upon which the AED was used and any monitors associated with the patient are associated with the incident in this way. In some embodiments, if the patient or user has pre-opted in then the data may be shared automatically. If the patient has not pre-opted, the user may request that access be granted.

In yet another example, a user may access the incident identifier and/or a bar code that embeds the incident identifier after the AED’s use has terminated to facilitate accessing the incident report and/or entering the incident identifier into other systems (e.g., a patient care record, a monitor record, etc.).

In another example, a web-based management platform (e.g., the platform described in U.S. Patent No. 12,594,431 which is incorporated by reference) may include an incident page that includes a GUI widget (e.g., an “add other data” button), that a user may utilize to identify a monitor whose data should be added to the incident report. The specific process used to integrate the data may vary widely. By way of example, in some implementations, selection of an Add Other Data button pulls up a list of integrated device types. When this is pulled up, a user can select a particular monitor type and prompt a login for that monitor management platform account which will cause the monitor management platform account to share data from the relevant monitor with the AED management server for inclusion in the incident report.

Turning next to FIG. 2B a representative process for creating integrated incidents reports will be described from the side of the management server 20/incident report server 50. In the embodiment described below, the management server and incident report server functionalities are combined in a single server infrastructure. However, in other embodiments, they may take the form of independent infrastructures with their respective duties assigned appropriately. When separated, the responsibilities assigned to each server may vary based on design goals and the two nodes can communicate back and forth as appropriate, but in the description below, the term management server tends to be used when the function is more of a management server function, and the term incident report server tends to be used more when the function is more of an incident report management function.

From the standpoint of the management server, the process starts when it receives an incident notification. Block 230. In the flow illustrated in FIG. 2B, that may take the form of receiving the first incident message sent by the AED in step 204. When the management/incident report server is notified of a new incident, it creates an incident record in AED database 22 and/or incident report database 52. Block 233. The incident record is used to store information about the incident and can be used to generate incident reports at any time. The incident notification includes the incident identifier (or other information that can be used to uniquely identify the incident), an AED identifier that uniquely identifies the active AED (e.g., a device serial number) and a timestamp indicating when the AED was activated. As previously mentioned, a concatenation of the AED identifier and the activation timestamp, or a hash of the two, may be used as an incident identifier to uniquely identify the incident.

Optionally, the incident report is made available to interested parties as soon as the incident record is created. Block 235. If desired, the management server can send notifications to potentially interested parties – such as the AED’s owner or administrator or other designated individuals or entities – at that point notifying them of the incident and/or the fact that they can access an incident report using the incident identifier. Regardless of whether any such notifications are sent, an interested party with knowledge of the incident identifier can obtain an incident report that has up to date information about the incident at any time after the incident record is first created. For example, in some circumstances, a responding EMT in route to the incident may be able to access the incident report while the AED is still in use to get some understanding of the incident prior to arrival at the incident scene.

The management server 20 has an AED database 22 which contains information about each of the managed AEDs. One of the fields in the database indicates any monitors that are associated with specific AEDs. When the management server learns of a new incident, it checks database 22 to determine whether there are any monitors that have been pre-associated with the AED. Block 238. If a monitor is pre-associated with the AED, then the server sends a request for pertinent monitor data to the monitor management server(s) 70 affiliated with the associated monitor. Block 240. If multiple monitors are associated with the AED, then such requests may be sent to the monitor management servers affiliated with each of the identified monitors. The monitor data request typically identifies the specific monitor(s) of interest, and indicates either explicitly or implicitly the desired monitor data. The type(s) and extent of information requested of / provided by the monitor management server will depend on the type of monitor involved as well as systems design goals regarding the exact types of information desired to be included in the in the incident report. In some embodiments, recent data detected by the monitor beginning “X” minutes before the AED was activated may be requested/provided. In some implementations/circumstances, the data request includes the AED’s activation timestamp from which time the monitor management server may determine the appropriate window of information to forward to the Incident Report Server 50. In others, the Incident Report Server may request data from a specific time window or from a designated time to the present. The amount of information that is deemed appropriate for inclusion in the incident report may vary based on the nature of the monitor and the expected medical usefulness of such information, but it is often useful to include monitor information starting 10 minutes or more before the AED was activated so that hopefully, the incident report shows the nature of the degradation of the patient into cardiac arrest. Although 10 minutes is given as an example, it should be appreciated that longer or shorter windows may be used in various implementations. In some circumstances, the monitor management server may have logic that analyzes the monitor data to determine (or extend) the window of time that is most appropriate to send.

In circumstances in which an AED scan for nearby monitors identified one or more nearby devices, the management server will receive the list of the detected devices from the AED. Some of the detected devices may be monitors with which the incident reporting system is integrated, while others may be other types of devices that have no medical applicability or integration with the incident reporting system. Thus, when the management server receives an indication of nearby devices (block 242), it will check to determine whether the device is a monitor which the incident reporting system has an integration with. Block 244. This can be accomplished by the AED management server checking an “integrated monitor list” which identifies the set of monitor types for which the AED management server has an integration with or is otherwise able to handle an automatic data pull. If a detected device is a monitor that supports integrated incident reports, a request is sent to the monitor management server requesting relevant data from the identified monitor (block 240) as previously described. With this arrangement, if the monitor device type (e.g. an Apple Watch) is detected, the AED management server is able to query the Apple Watch monitor management server with the device ID, and the monitor manager server is able to transfer back any applicable data collected by the monitor within X minutes (10 minutes, an hour, etc.) of the AED use timestamp. If it’s not immediately available and uploads later, the external monitor management server may have a way to initiate a “now I have data for you” message that prompts the AED management server to later incorporate the data, even if it’s not immediate.

It should be appreciated that there are a wide variety of devices that can obtain information relevant to a medical incident so there are a large number of potential integrations. Some of the most straight forward and highest need integrations involve wearable or implantable cardiac monitors (e.g., Holter monitors, ICMs, etc.) because such devices are typically prescribed when the patient is known to have an elevated risk of cardiac arrest. However, there are a number of other types of devices that can supply useful information as well. For example, smart watches and fitness trackers commonly track heart rates and some have the ability to record the wearer’s ECG and/or detect atrial fibrillation (AFib). For example, the Apple Watch Series 4 and up can record ECGs and detect AFib. Fitbit fitness trackers track heart rates and various models can also detect AFib. Of course there are many others, and it is expected that more products will have these functionalities in the future.

Pulse Oximeters typically measure pulse rate and blood oxygen saturation (SpO2) both of which are useful information in understanding events leading up to a cardiac arrest, as well as understanding the patient’s condition during an arrest.

Athletic heart rate monitors – such as chest strap heart rate monitors – track the athlete’s heart rate during workouts, which statistically, is a time of elevated risk of cardiac arrest – and simple heart rate information leading up to a cardiac arrest can be useful in better understanding the event.

There are a number of portable ECG/EKG devices that are designed for home use, and they may have useful information about the patient’s prior condition – even if they weren’t in use at the time of the cardiac arrest.

During and relatively shortly after an incident, the incident report server can expect to receive responses to its requests, as well as various updates and queries. For example, when the AED regularly sends current status messages and/or status updates, the incident report server will receive or be forwarded such updates. When a status update is received, the server updates the corresponding incident record in the incident database 52. Block 250.

At some point, the incident report server receives the requested monitor data from the monitor management server(s). In some circumstances, the monitor management server may only send a single report that includes all of the relevant information it has. In other circumstances (e.g., when the monitor continues to monitor the patient through a cardiac arrest incident while the AED is deployed and/or after the AED deployment has been terminated), the monitor management server may receive additional data from the identified monitor(s) after the initial response is sent. In the latter case, the monitor management server will periodically forward relevant monitor data to the incident report server.

It should be appreciated that the amount and nature of the monitor data that is deemed valuable enough to include in the incident record will vary based on the nature of the monitor, and different monitoring systems may have different latencies in obtaining data from their respective monitors, so the timing of when monitor data is received and whether, or how many updates are received can vary significantly between monitor providers and individual incidents. However, from the standpoint of the incident report server 50, any updates it receives that are pertinent to a particular incident are simply stored in the associated record. Block 252.

As pointed out above, the AED is preferably arranged to send an incident log to the management server at the conclusion of the AED’s use. The incident log often has relevant information beyond the information that the management server receives in real-time during use of the AED. For example, the incident log may include a full ECG from the time that the electrode pads are placed on the patient until the electrodes are removed from the patient, or the AED is deactivated. When the incident report server receives the incident log (or information from the incident log) it updates the record accordingly. Block 254. Although the full ECG is described as being part of the incident log, it should be appreciated that in other embodiments, the ECG may additionally or alternatively be streamed or transmitted in parts to the AED management server as during the incident. In such circumstances, the incident record and/or any viewed incident report may be updated as such information is received – which in some cases facilitates a 3^{rd} party (e.g., a doctor reviewing the incident) to see the ECG in real-time or near real-time during the incident.

At any time after the incident record is initially created (e.g., in block 233), an interested party may request access to an incident report. Interested parties may include a responding EMT, personnel at the emergency room facility that will receive the patient, other medical personnel that are, or will be attending to the patient, an electronic patient care records system, an administrator associated with the AED, etc.. If/when an incident report is requested, the desired report is generated based on the incident record as it then stands. The incident report can then be served, or otherwise delivered to the requestor. Block 256. A noteworthy feature of the described system is that incident reports can be obtained in real-time while the incident is still active, and the AED is still being used. This can be particularly helpful for EMTs responding to an incident and to medical personnel who are, or will be, involved in the patient’s initial emergency care.

Many of the use cases described above contemplate that the requestor of the integrated incident report is a medical provider or medical personnel. But it is worth mentioning that automatic or semi-automatic integrations with medical records systems (e.g., electronic patient care record (EPCR) systems) are very valuable as well so that the incident report is automatically or semi-automatically entered into the victim’s patient care record.

Referring next to FIG. 2C, a representative process from the perspective of the monitor management server 70 will be described. There are a wide variety of existing monitor management systems, and we don’t endeavor to try to describe them all here. However, some of the features relevant to incorporating independent monitor data into AED incident reports are described.

The monitor management server 70 will typically periodically receive patient data from their managed monitors. Block 272. The frequency of the updates, as well as the way that the updates are received can vary widely between systems, and indeed between different managed monitors within any particular system. Some monitors are configured to automatically send updates on a periodic basis – e.g., substantially continuously, hourly, daily, weekly etc.. Some automatically send updates when relevant events are detected. Some send updates when queried by the management server, or instructed by the patient or other appropriate individuals. Some provide updates when they are within range of a device that serves as a base station or relay. Some provide updates based on other factors. And many will provide data via multiple of these paths.

If/when the monitor management server receives a request for data for inclusion of a particular monitor’s data in an AED incident report (block 275), the monitor management server will obtain the requested data if it is not already in its possession. Block 277. Some monitor management systems are arranged so that the monitor management server 70 is continually fed patient status information from its associated monitors 75. In such systems/circumstances, the monitor management server 70 may immediately access and forward the requested data to the incident report server. In other systems/circumstances, the monitor management server will need to query the identified monitor to obtain the requested data. Either way, the monitor management server forwards the requested monitor data to the incident report server when the data is available. Block 279.

In some circumstances, the monitor will send the requested monitor data in a single batch. However, as previously described, one of the powerful features of the described system is that medical personnel and other authorized parties can view the incident in real time during the incident. Therefore, if/when the monitor has the ability to continue conveying monitor data to the monitor management server during and after the incident, the server may continue receiving new data and sending updates to the incident report server as long as the data is desired, and the relevant monitor is still collecting data. In various systems, the monitor management server 70 may continue to send updates for a predetermined amount of time, for a specific period requested by the incident report server, until it receives instructions to stop sending the requested information, or for any other suitable period. It should be apparent that the described system enables the incident report server to integrate such information into any incident reports it provides.

It is noted that in many circumstances, the monitor may continue to record data even after the AED is no longer being used. As previously mentioned, when EMTs reach the patient, they will typically connect the patient to an ACLS system, at which point the monitor’s data may no longer be useful. Therefore, in some embodiments, the incident report server will instruct the monitor management server to quit sending updates when the incident has terminated. However, if/when circumstances warrant, the monitor management server can continue to send updates from the monitor as long as such information is deemed useful.

In the embodiment described above with respect to FIGS. 2A-C, the two primary pathways for triggering monitor data integration are: (i) pre-associations of an AED with a monitor; and (ii) scanning for potential monitoring devices when an AED is deployed. There are several other pathways through which the integration can be triggered. For example, FIG. 3 illustrates a process triggered by a monitor system detecting or being informed that a defibrillator deployment has occurred.

Some monitors are considered defibrillation safe in that they will not be damaged, or cause injury to a patient even if they are in use monitoring the patient when a defibrillation shock is administered. By way of example, most implantable cardiac monitors (ICMs) and medical grade wearable cardiac monitors are defibrillation safe. As such, many of these monitors can readily be designed to detect the delivery of a defibrillation shock. Monitors in contact with the patient, and especially with the patient’s thorax, can readily detect the delivery of an external defibrillation shock because of the very high voltages utilized in such shocks. But a monitor can also be designed to detect the connection of a defibrillator to the monitored patient even before a defibrillation shock is delivered and/or even if no shock is ever delivered. For example, when an AED’s defibrillation electrode pads are placed on a patient, the AED will typically check the impedance between the electrode pads by causing a small electrical current to flow between the pads. This current, which is fairly unique, can be detected and used to infer that pads have been placed on the patient. Of course, monitors may be configured to detect the deployment of a defibrillator in other ways. For example, cameras and/or other sensors can be used to detect pad placement. In another example, some AEDs are configured to wirelessly broadcast status messages as advertisement when they are in use. Thus, a monitor can be designed to detect such messages and determine that a nearby defibrillator has been deployed based on such messages. By way of example, U.S. Patent No. 11,452,881 (P016A) and 11,077,312 (P016B), each of which are incorporated herein by reference describe such defibrillators. Alternatively, deployment of the AED may prompt a connection with a monitor, or a device associated with the monitor, through which the monitor system may be informed of the deployment. Regardless of the specific mechanisms that are used, the process of FIG. 3 can be triggered when a monitor (or a device associated with a monitor) detects deployment of a defibrillator. (Block 301).

In other circumstances the monitor system (e.g., the monitor itself, or an app or other device associated with a monitor) may have a user interface that allows users or people affiliated with the patient (e.g., family members, care givers, co-workers etc.) to input an incident ID or other relevant information at the time of the incident, or after the incident, to indicate that an AED has been used on a patient associated with the monitor. Some AEDs are configured to display an incident ID when they are finished being used and/or at other appropriate times. Such a system is described, for example, in U.S. Patent Application No. 17/217,738 (P022), which is incorporated herein by reference. This incident ID can be entered into the monitor system to trigger the data integration process of FIG. 3. Block 302.

When the monitor system detects or is informed of a defibrillator deployment, the monitor (or a device associated with the monitor) sends a defibrillator deployment message to the monitor management server. Block 304. The information contained in the defibrillator deployment message will vary based on the capabilities and knowledge of the monitor. As previously mentioned, monitors can readily be configured to detect activation of a nearby AED, as for example by detecting BLE advertisements containing status messages that are broadcast by the AED in association with a use of the AED. In some embodiments, the defibrillator deployment message includes one or more of: (i) a monitor identifier that identifies the detecting monitor system (e.g., the monitor’s serial number); (ii) a timestamp indicating the time that the deployment was detected, or the monitor was otherwise informed of the incident; (iii) the location of the monitor; (iv) an indication of how the deployment was detected; (v) the incident ID (if/when known); and (vi) any other relevant information that is available to the monitor.

The additional information that is available to the monitor system will vary based upon how the monitor system learns of the deployment. For example, when the deployment detection is based on the detection of a status message broadcast by the AED, the available information will include any and all information contained in the status message (which may include information such as the incident ID; the AED’s ID; various timestamps indicating the timing of different events – e.g., timestamp indicating when the AED was powered on, when the electrode pads were placed, etc.; indications of whether or not a defibrillation shock is advised; timestamps of when any shock/no shock decisions were made; and an indication of the number of shocks that have been delivered (if any) and timestamps indicating when such shock(s) were delivered; ECG segments, etc.). When deployment detection is based on the detection of the delivery of a defibrillation shock, the monitor will know the exact time of the shock delivery. When the monitor detects placement of electrode pads, it will know the time the pad placement was detected. Any of this type of information that is available to the monitor system is preferably included in the defibrillator deployment message.

Similarly, when the monitor system is informed of the deployment by a user or bystander manually inputting or scanning an incident identifier into the monitor system, at a minimum, the incident identifier is known. When the monitor system receives an incident status message from a deployed AED, the monitor system will likely know the incident identifier, as well as other relevant information such as the AED’s serial number, the time the AED was first activated, etc. Again, additional information that is available to the monitor system is preferably included in the defibrillator deployment message. For example, when the monitor system receives an incident status message from the AED, the full status message is preferably included in the defibrillator deployment message.

The monitor system also forwards the relevant monitor data to the monitor management server. Block 307. Depending on the system, the monitor data may be sent to the monitor management server together with, or in conjunction with the defibrillator deployment message, or simply in the normal course.

When the monitor management server 70 receives a defibrillator deployment message, it sends an incident query to the incident report management server 50 that informs server 50 of the incident. Block 310. Typically, the incident query will include (as available), the same types of information as the received defibrillator deployment message, plus any additional relevant information that the monitor management server 70 may have. For example, the monitor management server 70 may be aware of an AED that is associated with the monitor, and if so, the identity of the associated AED is preferably provided as well.

When the incident report server 50 receives an incident query (block 312), it checks to see whether it is aware of a corresponding incident. Block 315. The nature of this check will vary based on the information that is available in the incident query. In situations where the incident ID is known, the check is simple. In situations where the identity (e.g., serial number) of the deployed AED is known, or is suspected (e.g., based on a particular AED being associated with the detecting monitor system), the incident report management server can simply search its incident record database 52 for a record associated with the use of the designated AED at the time and/or location that the deployment was detected.

In circumstances where neither an incident ID nor the AED’s identity is known or there is no corresponding record associated with a suspected AED, the incident report management server performs a more general search of the incident record database 52 based on the information that is available. Records in incident records database 52 will generally include the location of the incident (as reported by the AED) and the time of the incident, including timestamps for various events including activation of the AED, deactivation of the AED, and various events in between, including pad placement and any shock delivery timestamps. Thus, the incident records database 52 can readily be searched for an incident that is/was occurring at the time and location identified in the incident query.

When the incident report management server identifies an incident record that corresponds to the incident indicated in the incident query (the “Yes” branch from block 315) it sends a monitor data request to the monitor management server – block 318 – which may be the same, or substantially the same, as monitor data request 240 discussed above with respect to FIG. 2B. The incident report management server knows which monitor to request data for because the relevant monitor was identified in the incident query. The monitor management server then forwards the requested monitor data (block 321) and updates thereto as appropriate. From there, essentially the same process described above with respect to FIGS. 2B and 2C may be followed integrate the received monitor data into the incident record and provided incident reports that integrate both AED and independent monitor data.

Several different approaches can be followed if/when the incident report management server is not able to identify an incident record corresponding to the defibrillator deployment detected by the monitor system in step 315. In some cases, the incident report management server may do nothing further because it doesn’t have an incident that correlates to the detected deployment. This could be because the deployed AED isn’t integrated with the described incident reporting system, or it could be that the “ defibrillator deployment” believed to have been detected by the monitor system was actually a false alarm, etc. There is also a chance that the monitoring network simply sent its incident notification to the incident report management server more quickly than the AED was able to. That can be due to a wide variety of factors including: (i) the connectivity of the treating AED at the location at which it is deployed; and (ii) the timing of when the AED sends information to the AED management server (e.g., some AEDs may be configured to only send incident logs, reports, status messages or other information after the event has ended), etc.

Therefore, in some embodiments, the incident report management server will create an incident record based one the incident query (block 331) if it is unable to identify an existing record corresponding to the event. Then, if an incident notification or other communication is later received from an AED that corresponds to the same event, the two can be associated at that time. Similarly, if two (or more) monitor systems from different monitor networks detect the same event – the incident management can associate the two (as well as any AED data) based on the time and location of the event, and/or other available information.

To Applicant’s knowledge, their AEDs are the first commercially available medical devices (much less AEDs) that have been integrated with Public Safety Answering Points (PSAPs) (e.g., 911 call centers in the United States), such that a PSAP can initiate communication with the device. This has led to an integration of their AEDs with emergency response systems at a deeper level than conventional monitoring systems enjoy. Over time, this is expected to familiarize various emergency services providers with Applicant’s incident reporting framework. Monitor providers can benefit from this framework by adopting incident notification processes similar to those described herein and in the incorporated U.S. Patent Application No. 17/217,738 (P022). Thus, even when there is no corresponding AED deployment, the described system can be used to create incident reports that contain only monitor data, and/or to integrate data from two or more independent monitors as there are many circumstances where such data alone may be very valuable to medical providers.

Referring next to FIG. 4, another mechanism for triggering report integration is described. Individuals associated with an AED - such as the AED owner, administrator, family members, or other authorized persons – will often have access to an AED management platform via a web browser or other suitable user interface. By way of example, a representative AED management platform is described in U.S. Patent No. 12,594,431 (P027C), which is incorporated herein by reference.

In some embodiments, the management platform includes an incident tab through which users can see any and all incidents associated with their AEDs. Selection of a particular incident brings up an incident details page that provides information about the incident and a GUI widget through which the full incident report can be obtained (e.g., a download Incident Report button). The incident details page (or any other suitable page within the management platform) also includes a Monitor Association GUI widget (not shown) that can be used to associate a particular monitor with the incident. When an interested party knows that the victim was wearing, had, or was otherwise under the view of a monitor, they can use the Monitor Association GUI widget to input the monitor ID of the victim’s monitor to thereby link the monitor to an incident. Block 410. After receiving the monitor ID, the management platform transmits a report integration request to the incident report server. Block 415.

When the incident report server 50 receives a report integration request it sends a monitor data request to the monitor management server 70 requesting the monitor’s data – which is step 240 of FIG. 2B described above and the monitor integration then proceeds as previously described.

Similarly, the monitor may be associated with an incident through a monitor management platform. That is, individuals associated with a monitor may access a corresponding monitor management platform through a web browser or other suitable user interface. The management platform includes an incident widget associated with the patient through which the incident ID provided by the AED can be entered to thereby associate the patient/patient monitor with the incident. After receiving the incident ID, the monitor management platform transmits an incident query to the incident report server – which is step 310 of FIG. 3 described above and the monitor integration then proceeds as previously described.

Referring next to FIG. 5, yet another mechanism for triggering report integration will be described. In some embodiments, a set of one or more individuals that are considered most likely to be treated by a particular AED are associated with that AED in what is referred to herein as a patient list. For example, in the context of a home AED, the associated individuals may be the family members. In the context of an office or business establishment, the associated individuals may be employees that are known to be of particular risk, or those wearing monitors or those interested in the program. In the context of an AED in a multi-family residential unit or a neighborhood, it may include individuals living in the complex or neighborhood. In the context of a residential care facility, it may include the residents of the facility. Of course, these are just examples and the size and nature of the group of people associated with a particular AED may vary widely.

When an AED having an associated patient list is used, it will render the patient list on its display screen after its use terminates. Block 504. This allows users who know the victim to select the victim from the patient list. Block 509. The AED then transmits the patient ID to the AED management server 20. Block 514. The AED management server uses the patient ID to look up whether the identified patient has any associated monitors. Block 519. If so, the logic flows to step 240 of FIG. 2B where the incident report server sends a monitor data request to the monitor management server(s) requesting the relevant data from the identified monitor(s). From there, the patient’s monitor data can be integrated with the AED incident report as previously described.

### Incident Report

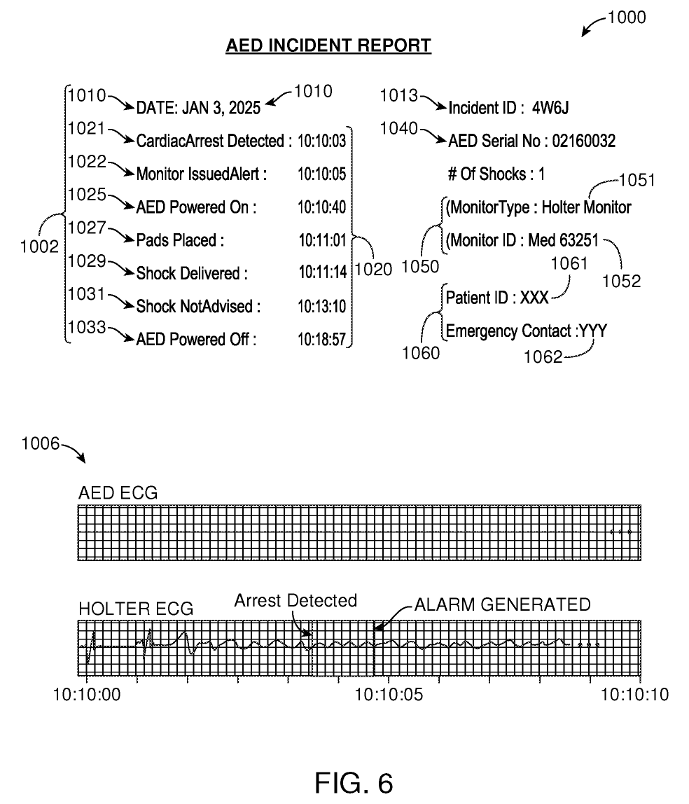

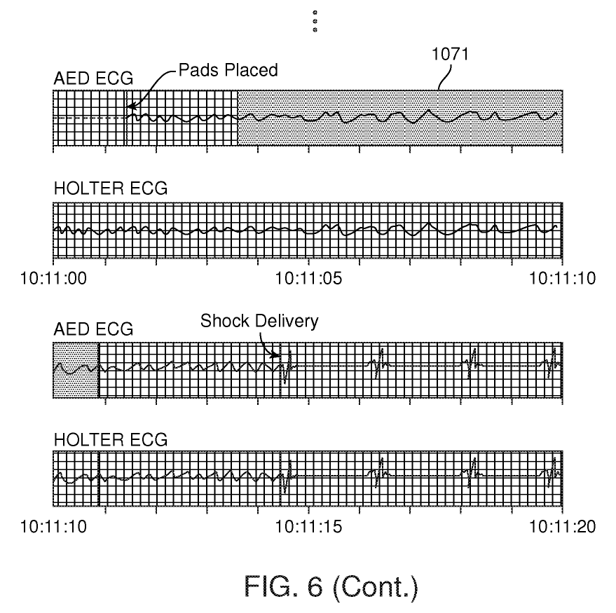

FIG. 6 illustrates a representative incident report that presents data from an independent monitor together with AED incident data. In the illustrated embodiment, the independent monitor is a Holter monitor that has the ability to detect cardiac arrests and issue alerts if/when abnormal heart rhythms (including cardiac arrests) are detected. Although a particular report style is described, it should be appreciated that the content and layout of the incident report may be widely varied based on factors such as the monitor whose data is incorporated, the AED information that is available and deemed to be of interest, etc.

The illustrated incident report 1000 includes an incident information section 1002 and an ECG section 1006. The incident information section 1002 includes a number of fields and entries that provide useful information about the incident. In the illustrated embodiments, the incident information includes a Date field 1010, an Incident ID field 1013, an event log 1020, an AED identification section 1040, a monitor identification section 1050, an optional patient identification section 1060.

Many of the described fields are self-explanatory. For example, the Date field 1010 includes a date identifier 1011 that indicates the date of the incident. The incident ID field 1013 presents the Incident ID. The event log 1020 presents timestamps associated with a series of events that occurred during the incident. The specific events shown will vary based on the nature of the event and the information that is available to the incident report server 50. It may include events detected by both the AED 10 that was used and any available monitor(s) 75. In the example shown, the monitor was able to detect the occurrence of the cardiac arrest and issue an alarm responsive to that detection. Thus, the first two event timestamps 1021 and 1022 present the time that the cardiac arrest was first detected and the time that the alert was generated respectively.

The event log 1020 further includes a number of timestamps of relevant events that the AED reported. In the example shown, these include: (i) an AED powered on timestamp 1025 that indicates the time the AED was turned on; (ii) a pads placed timestamp 1027 that indicates when the AED detected that the AEDs electrode pads had been placed on the patient; (iii) a shock delivered timestamp 1029 that indicates when a defibrillation shock was delivered – if more than one shock were delivered, timestamps would be provided for each shock delivered; (iv) a shock not advised timestamp 1031 that indicates the timing of a classification performed by the AED that resulted in the AED determining that a defibrillation shock was not advised – again, more than one such shock not advised timestamps may be provided if/as appropriate; and (v) a powered down timestamp 1033 that indicates when the AED was turned off. It should be apparent that specific information presented in the event log 1020 may vary widely based on the information that is made available to the incident report server and deemed appropriate for inclusion in the report. For example, either the AED or the monitor may have a variety of other event information worthy of presentation. In another example, U.S. Patent Publication Nos. 2024-0366953 (P027A) and 2024-0371511 (P027B), each of which are incorporated herein by reference, describe incident reports that include timestamps for events performed by a PSAP (e.g., a 911 call center) that handled an emergency call related to the incident and/or managed the dispatch of emergency response personnel to the incident. When available, timestamps for such emergency response related events can be included in the incident report.

The incident report may include a variety of other information reported directly or indirectly by either the AED or the monitor. For example, in the illustrated embodiment, Shocks Field 1038 is effectively a counter that identifies the number of defibrillation shock delivered by the AED during the incident.

The AED identification section 1040 identifies the AED used in the incident. In the illustrated embodiment, the AED is identified by way of its serial number, although it should be apparent that the AED can be identified in any desired way. In other embodiments, the AED may not be directly identified at all.

The monitor identification section 1050 identifies the monitor(s) whose data are included in the report. In the illustrated embodiment, the monitor is identified by way of its type (monitor type field 1051) and serial number (monitor serial number field 1053). Again, it should be apparent that the monitor can be identified in any desired way. If/when data from multiple monitors is included in the incident report, each of the reporting monitors may be identified in section 1050.

When a particular patient is associated with one or both of the monitor and the AED, it is possible to include selected patient information in the incident report. In the illustrated embodiment, relatively simple patient information in the form of the patient’s name or ID 1061 and emergency contact information (e.g., name and phone number of the patient’s designated emergency contact) are provided. However, it should be appreciated that in other embodiments, any amount of patient information that is pre-provided to either the AED management server 20 or the monitor management server 70 may be included. This can include the patient’s relevant medical conditions/history, medicines taken, the patient’s pre-event doctor (e.g., a cardiologist who prescribed the monitor), etc. Preferably, the patient related data would only be included on an opt-in basis.

In the illustrated example, both the AED 10 and the Holter monitor record the patient’s ECG. Thus, the ECG section 1006 presents both ECGs in an aligned manner. In the embodiment shown, a graph showing an ECG segment detected by the AED is presented first and the corresponding graph segment of the Holter monitor’s ECG is presented immediately below the AED’s ECG. Of course, if the monitor detects something in addition to and/or in place of the patient’s ECG, time corresponding segments of the detected data can be presented in a similar manner. Similarly, the example shows data from just two devices (i.e., the AED and the Holter monitor), but there may be circumstances in which data from two or more different monitors may be available. In such circumstances, the incident report may include data from the AED and multiple monitors, which would result in the presentation of data from 3, 4, 5 or more different devices in parallel rather than just two.

In some embodiments, the graphs are labeled with significant events that are detected by the AED and/or monitor to simplify reading. For example, some or all of the events presented in the event log may be marked at the appropriate locations in the graph. Similarly, other information such as the segment of the patient’s ECG 1071 that was used by the AED’s classifier to determine whether or not to deliver a defibrillation shock may be marked (as shown as shaded in FIG. 6). It is noted that FIG. 6 illustrates one data presentation style, but it should be appreciated that the data from one or both of the AED and the monitor may be presented in a variety of different ways.

### Automated External Defibrillator

In some embodiments, the AED 10 includes a base defibrillator unit 110 having an interface unit 200 secured thereon. In other embodiments, the functionalities of the base unit and interface unit described below are combined into a single unit or distributed among more than two associated modules.

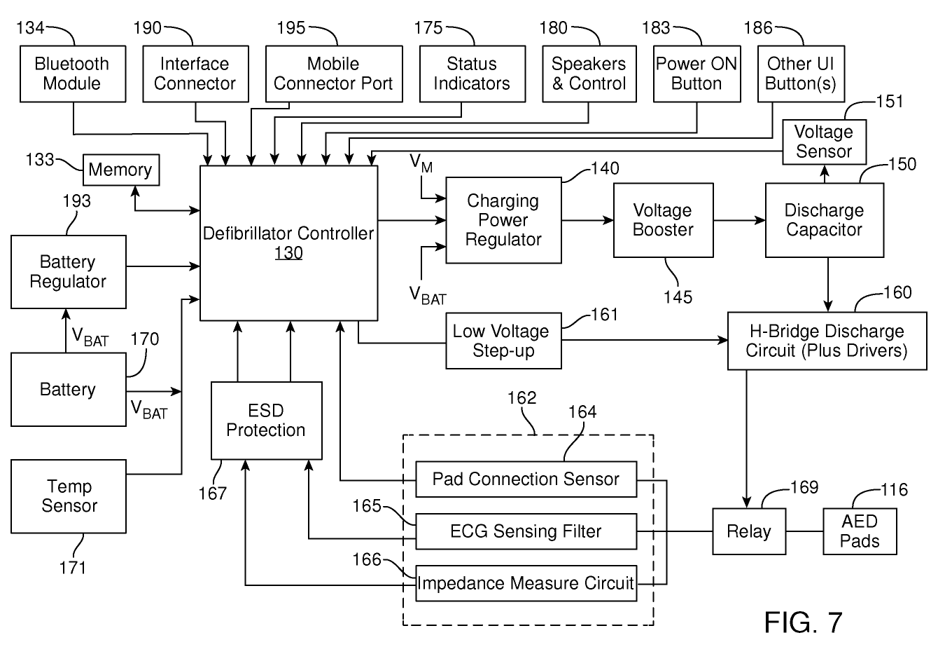

FIG. 7 is a block diagram illustrating one representative electronics control architecture and associated components suitable for use in the base defibrillator unit 110. In the illustrated embodiment, the electronic components include a defibrillator controller 130, memory 133, a wireless communications module in the form of Bluetooth module 134, a charging power regulator 140, a voltage booster 145 (which may have multiple stages), a high voltage capacitor 150 for temporarily storing sufficient electrical energy suitable to provide a defibrillation shock, discharge control circuitry 160, pad related sensing circuitry 162 and relays 169, power storage unit 170, battery regulator 193, status indicator(s) 175, speaker(s) 180 and one or more electrical connectors (e.g., interface connector 190, mobile connector port 195, charger connector (not shown), etc.). The charging power regulator 140 and voltage booster 145 which cooperate to control the charging of the shock discharge capacitor 150 are sometimes referred to herein as a charging circuit.

The defibrillator controller 130 is configured to control the operation of the base defibrillator unit and to direct communications with external devices, as appropriate. In some embodiments, the defibrillator controller includes one or more processors arranged to execute software (some or all of which may take the form of firmware) having programmed instructions for controlling the operation of the base unit, directing interactions with a user and communications with external components. The software may be installed on the memory 133. Although the singular term memory is often used herein, it should be appreciated that the memory may be divided into multiple different parts which take any suitable form or combination of forms (e.g., various types of RAM, ROM, PROM, EEPROM, etc.) Unless the context suggests otherwise, references to “memory” herein are intended to cover all suitable forms and combinations of physical memory. Similarly, although the singular term “processor” is often used herein, it should be appreciated that any appropriate number of processors and/or processing cores can be utilized and unless the context suggests otherwise, references to “processor” herein are intended to cover processing units composed of one or more physical processors or processing cores.

The base defibrillator unit 110 may optionally be configured so that it is capable of drawing power from certain other available power sources beyond power storage unit 170 to expedite the charging of shock discharge capacitor 150. The charging power regulator 140 is configured to manage the current draws that supply the voltage booster, regardless of where that power may originate from. For example, in some embodiments, supplemental power may be supplied from a mobile device coupled to mobile connector port 195 or from a portable charger/supplemental battery pack coupled to charger connector 197.

The voltage booster 145 is arranged to boost the voltage from the operational voltage of power storage unit 170 to the desired operational voltage of the discharge capacitor 150, which in the described embodiment may be on the order of approximately 1400V-2000V (although the defibrillator may be designed to attain any desired voltage). In some embodiments, the boost is accomplished in a single stage, whereas in other embodiments, a multistage boost converter is used. A few representative boost converters are described in the incorporated U.S. Patent No. 10,029,109. By way of example, in some embodiments, a flyback converter, as for example, a valley switching flyback converter may be used as the voltage booster 145 – although it should be appreciated that in other embodiments, a wide variety of other types of voltage boosters can be used.

A voltage sensor 151 is provided to read the voltage of the capacitor 150. The voltage sensor 151 may take the form of a voltage divider or any other suitable form. This capacitor voltage reading is utilized to determine when the shock discharge capacitor 150 is charged suitably for use. The sensed voltage is provided to controller 130 which determines when the capacitor 150 is charged sufficiently to deliver a defibrillation shock. The capacitor 150 can be charged to any desired level. This can be useful because different defibrillation protocols advise different voltage and/or energy level shocks for different conditions. Furthermore, if the initial shock is not sufficient to restart a normal cardiac rhythm, some recommended treatment protocols call for the use of progressively higher energy impulses in subsequently administered shocks (up to a point).

The discharge circuitry 160 may take a wide variety of different forms. In some embodiments, the discharge circuitry 160 includes an H-bridge along with the drivers that drive the H-bridge switches. The drivers are directed by defibrillator controller 130. The H-bridge outputs a biphasic (or other multi-phasic) shock to patient electrode pads 116 through relays 169. The relays 169 are configured to switch between an ECG detection mode in which the patient electrode pads 116 are coupled to the pad related sensing circuitry 162, and a shock delivery mode in which the patient electrode pads 116 are connected to H-Bridge to facilitate delivery of a defibrillation shock to the patient. Although specific components are described, it should be appreciated that their respective functionalities may be provided by a variety of other circuits.

The pad related sensing circuitry 162 may include a variety of different functions. By way of example, this may optionally include a pad connection sensor 164, ECG sensing/filtering circuitry 165 and impedance measurement chip/block 166. The pad connection sensor is arranged to detect that the pads are actually connected to (plugged into) the base defibrillator unit 110. The ECG sensing/filtering circuitry 165 senses electrical activity of the patient’s heart when the pads are attached to a patient. The filtered signal is then passed to defibrillator controller 130 for analysis to determine whether the detected cardiac rhythm indicates a condition that is a candidate to be treated by the administration of an electrical shock (i.e., whether the rhythm is a shockable rhythm) and the nature of the recommended shock. When a shockable rhythm is detected, the controller 130 directs the user appropriately and controls the shock delivery by directing the H-bridge drivers appropriately.

In some embodiments, the power storage unit 170 takes the form of one or more batteries such as rechargeable Lithium based batteries including Lithium-ion and other Lithium based chemistries, although other power storage devices such as one or more supercapacitors, ultracapacitors, etc. and/or other battery chemistries and/or combinations thereof may be used as deemed appropriate for any particular application. The power storage unit 170 is preferably rechargeable and may be recharged via any of a variety of charging mechanisms. In some embodiments, the power storage unit 170 takes the form of a rechargeable battery. For convenience and simplicity, in much of the description below, we refer to the power storage unit 170 as a rechargeable battery. However, it should be appreciated that other types of power storage devices can readily be substituted for the battery. Also, the singular term “battery” is often used, and it should be appreciated that the battery may be a unit composed of a single battery or a plurality of individual batteries and/or may comprise one or more other power storage components and/or combinations of different power storage units.

In some embodiments, the base defibrillator unit 110 is capable of drawing power from other available power sources for the purpose of one or both of (a) expediting the charging of shock discharge capacitor 150 and (b) recharging the power storage unit 170. In some embodiments, the battery can be recharged using one or more of the externally accessible connector ports 195, a dedicated charging station, a supplemental battery pack (portable charger), an interface unit 200, etc. as will be described in more detail below. When wireless charging is supported, the base defibrillator unit may include a wireless charging module 174 configured to facilitate inductive charging of the power storage unit 170 (e.g., using an inductive charging station 294, or other devices that support inductive charging, as for example an inductively charging battery pack, a cell phone with inductive charging capabilities, etc.).

The base unit also includes a number of software or firmware control algorithms installed in memory 133 and executable on the defibrillator controller. The control algorithms have programmed instructions suitable for controlling operation of the base unit and for coordinating the described broadcasts, as well as any point-to-point communications between the base unit 110 and the interface unit 200, connected devices, and/or any other attached or connected (wirelessly or wired) devices. These control routines include (but are not limited to): communication control algorithms, heart rhythm classification algorithms suitable for identifying shockable rhythms; capacitor charge management algorithms for managing the charging of the discharge capacitor; capacitor discharge management algorithms for managing the delivery of a shock as necessary; user interface management algorithms for managing the user instructions given by the defibrillator and/or any connected user interface devices (e.g. interface unit 200, mobile communication device 105) during an emergency; battery charge control algorithms for managing the charging of power storage unit 170; testing and reporting algorithms for managing and reporting self-testing of the base unit; software update control algorithms and verification files that facilitate software updates and the verification of the same.

In many installations, an AED will be expected to be stored for long periods of time without being plugged into power and therefore relying solely on battery power. Accordingly, it is important to minimize the power drain as much as possible during this time. In some embodiments, the defibrillator controller is configured to shut down power to all electrical components (including itself) except for (a) a real time clock (not shown), (b) the Bluetooth module 134, and (c) a power controller (not shown) when the AED is in the standby (resting) mode. In some embodiments, the clock and/or the power controller are integrated into the Bluetooth module 134 or an I/O adaptor board that includes the Bluetooth module. The clock is configured to periodically send a status check alarm to the power controller which wakes the power controller up from a sleep mode when the power controller is separate from the Bluetooth module. In response to the status check alarm, the power controller restores power to the entire system. Once power is restored, the defibrillator controller 130 performs a status check and instructs the Bluetooth module 134 to update the standby status message 150 as appropriate. After the status check is performed, the defibrillator controller will again shut down power to all electrical components except the clock, the Bluetooth module 134 and the power controller. The status check alarms can be generated at any desired intervals, as for example, once each day.

With the described power management scheme, the Bluetooth module 134 can continue to broadcast standby status messages even when the AED is powered down.

If the Bluetooth module 134 makes a connection while the AED is in the standby mode, it sends a message to the power controller, which in turn, powers on the system, thereby allowing the defibrillator controller or other suitable component to communicate with the connecting device. Thus, the AED can effectively be woken up via a Bluetooth connection request. Of course, the power controller is also configured to power the system when a user presses the AED’s “ON” button or otherwise activates the AED.

In other embodiments, one or more of the electrical components of the AED, such as the defibrillator controller 130, can be placed in a “sleep” mode rather than being turned off when the AED is in the standby mode. However, a significant advantage of actually turning the defibrillator controller and the bulk of the AED’s electrical components off as opposed to placing them in a sleep mode is that it eliminates the power draw associated with the sleep mode, thereby potentially extending the AED’s shelf life. It should be appreciated that the power controller / power down approach can be also used with AEDs that don’t incorporate a Bluetooth module and/or support the low energy broadcasts described herein.

In some embodiments, a temperature sensor 171 is provided within the defibrillator itself for detecting the internal temperatures of the AED (as opposed to an environmental temperature), and is used to trigger temperature fault notification and clearance messages. Preferably, the temperature sensor is positioned adjacent one of the more temperature sensitive components such as battery 170 so that the reported temperature is directly related to the internal temperature of the AED near the temperature sensitive component.

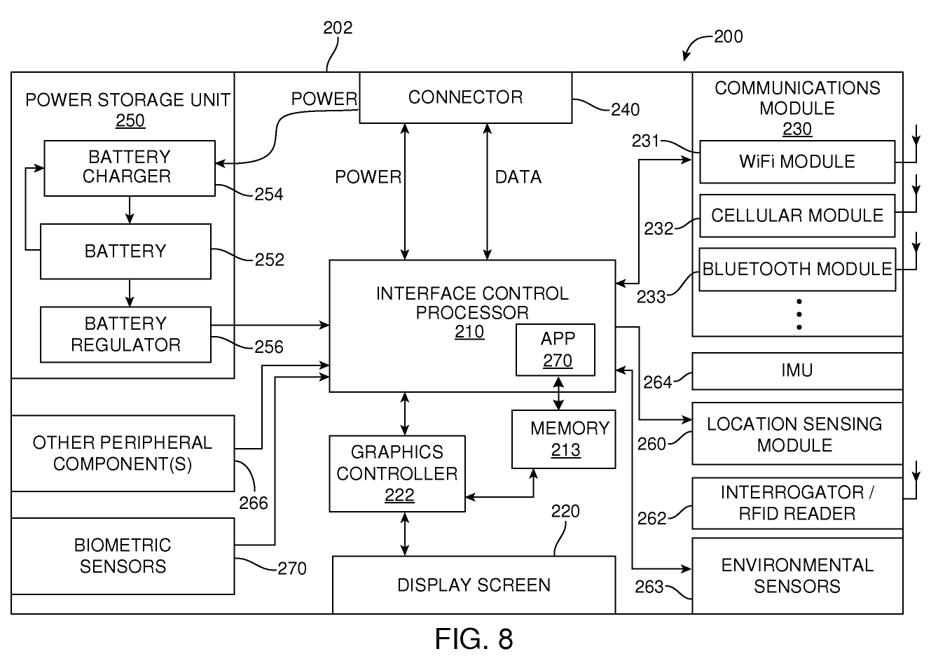

FIG. 8 illustrates some of the electrical components of a representative interface unit 200. In the illustrated embodiment, the interface unit 200 includes an interface controller (processor) 210, memory 213, a display screen 220, a communications module 230, an electrical connector 240, an interface unit power storage unit 250, and a location sensing module 260, an interrogator 262, and an inertial measurement unit 264, all of which may be housed within the interface unit housing 202. The interface unit has software (which may take the form of firmware and/or an app 270) installed or installable in memory 213 to provide programmed instructions suitable for controlling operation of the interface unit and for coordinating communications between the interface unit 200 and the base defibrillation unit 110 and/or remote devices to provide the described functionalities of the interface unit.

The interface unit controller (processor) 210 controls operation of the interface unit and coordinates communications with both the base unit 110 and remote devices such as a central server (as will be described in more detail below). In some embodiments, the processor 210 is arranged to execute a defibrillator app 270 or other software that can be used both during use of the defibrillator system 100 during a cardiac arrest incident and to facilitate non-emergency monitoring or/or use of the defibrillator system 100. Similar to the base unit processor 130 discussed above, unless the context suggests otherwise, the processor 210 may take the form of a single processor, multiple processors, multiple processing cores and other processing unit configurations.

The display screen 220 is a touch sensitive screen suitable for displaying text, graphics and/or video under the direction of the processor 210 to assist both during both emergency situations and at other times. The touch sensitive screen is configured to receive inputs based on a graphical user interface displayed thereon. In some embodiments an optional graphics controller 222 may be provided to facilitate communications between the interface control processor 210 and the display screen 220. In other embodiments, the functionalities of the graphics controller may be part of the processor 210.

The communication module 230 is provided to facilitate communications with remotely located devices such as the central server. The communications module 230 may be configured to utilize any suitable communications technology or combination of communication technologies including one or more of cellular communications, Wi-Fi, satellite communications, Bluetooth, NFC (Near Field Communications), Zigbee communications, DSRC (Dedicated Short-Range Communications) or any other now existing or later developed communications channels using any suitable communication protocol. By way of example, in the illustrated embodiment, the communications module 230 includes Wi-Fi, cellular and Bluetooth modules 231, 232 and 233 that facilitate Wi-Fi, cellular and Bluetooth communications respectively.

The electrical connector 240 is configured to mate with interface connector 190 on the base defibrillator unit 110. The connectors 190 and 240 are configured to facilitate communications between the defibrillator controller 130 and the interface unit’s processor 210. The connectors 190 and 240 are also preferably arranged to supply power from the interface unit 200 to the base unit 110 as will be described in more detail below. In some embodiments, power will only be provided in one direction – i.e., from the interface unit 200 to the base unit 110 and not in the reverse direction during operation. A good reason for this approach is that the defibrillator is the most important component from a safety standpoint, and it is often undesirable to draw power from the base unit to power other devices (including the interface unit 200) in a manner that could reduce the energy available to charge the discharge capacitor in the event of an emergency. However, in some embodiments, the power supply may be bi-directional (at least in some circumstance) if desired – as for example if the base unit is not in use, is fully charged and plugged into an external charging power supply, etc.; or if the power passed to the interface unit is not coming from the base unit’s internal battery (e.g., it is coming from a charger, a mobile communication device, or other device connected or attached to the base unit), etc..

The connectors 190 and 240 can take a variety of forms. They can be connectors with accompanying transceivers configured to handle processor level communications (such as UART, SPI, or I2C transceivers), with additional pins for power delivery (Power + GND), and connection verification (i.e. a pin that detects when there is a connection between the interface unit and the Base AED and triggers an interrupt on the Base AED signifying that there is not a unit connected). They can also be more standardized connectors such as USB connectors.

The interface power storage unit 250 provides power to operate the interface unit 200. In many embodiments, the power storage unit takes the form of a battery 252 with associated control components, although again a variety of other power storage technologies such as supercapacitors, ultracapacitors, etc. may be used in other embodiments. The associated control components may include components such as a battery charger and maintainer 254, which may include various safety monitors, and battery regulator 256. Preferably, the power storage unit 250 is rechargeable, although that is not a requirement. In some embodiments, the power storage unit 250 may also be arranged to supply supplemental power to the base unit 110. Depending on the structure and/or state of the base unit, the supplemental power can be used to help charge the discharge capacitor 150 during use; to power or provide supplemental power for the defibrillator electronics and/or to charge the base defibrillator unit’s power storage unit 170. In other embodiments, a supplemental battery within the interface unit (not shown) may be used to provide the supplemental power for the base unit rather than the power storage unit 250.

The location sensing module may incorporate a variety of technologies including Global Navigation Satellite Systems (GNSS) (e.g., GPS), Wi-Fi positioning, cellular triangulation, assisted GPS, Bluetooth Beacons, Near-Field Communications (NFC) and/or other location determining technologies. When requested, the interface unit can report its current location based on the location sensing technology that is believed to have the best accuracy under the then-present circumstances.

The interrogator 262 is a device that queries or scans for nearby transponders/tags. By way of example, RFID readers work well for this purpose. In other embodiments, the scanning function of the interrogator may be performed by, or supplemented by, components of the communications module 230. For example, the Bluetooth module 233 may be configured to perform BLE scans, or more generally, Bluetooth scans for nearby devices. When other communications modules are used (e.g., NFC modules, Wi-Fi modules, Zigbee modules, Z-Wave modules, DSRC modules, etc.), those modules may perform similar scans.

The inertial measurement unit (IMU) is a motion sensing system that can be used to help detect movement of the AED. The IMU can be useful for a number of purposes, including triggering activation of the AED if/when the AED is physically moved.

The interface unit may also optionally include various environmental sensors 263 and other peripheral components 266. When desired, the interface unit may include any of a wide variety of different types of sensors and peripheral components. For example, in selected embodiments, the interface unit may include one or more accelerometers and/or gyroscopes, a temperature sensor, a humidity sensor, a time of day sensor or clock, or any other desired sensors or components.

The interface unit 200 is preferably configured to securely mechanically attach to the base unit 110. Typically, the interface unit is detachable such that it may be separated from the base unit if desired – although in other embodiments, the attachment may be more permanent in nature. The specific mechanical attachment utilized may vary widely in accordance with the needs of any particular embodiment. In some embodiments, press or form fitting attachment structures are used, while in others, latch and catch mechanisms, snap fit structures, etc. are utilized alone or in combination to releasably attach the interface unit to the base. However, it should be appreciated that a wide variety of other structures can be used in other embodiments. In some embodiments, the interface unit includes an attachment sensor (not shown) that senses when the interface unit is attached to a base unit.

In some embodiments, the interface unit may also include one or more biometric sensors 270. The biometric sensors may vary based on the needs of any particular defibrillator. Some of the biometric sensors may be suitable for use in detecting or evaluating CPR performed during emergency use of the defibrillator. Other biometrics may be useful in more general health management applications. For example, in some embodiments, the biometric sensors may include one or more of a pulse or heart rhythm sensor, a blood pressure sensor, a glucose monitor, a pulse oximeter, an ECG monitor, a sleep tracker, a thermometer, etc.

A benefit of the described modular defibrillator architecture is that the interface unit can be (and preferably is) designed to provide robust connectivity, effectively making the defibrillator a highly connected device. The relatively large touch sensitive display screen provides an interface that can be easily used by users of most any age. The dedicated interface unit processor(s) and corresponding memory allow the interface unit to be programmed to provide a number of functionalities that are not available in defibrillators that are commercially available today. Notably, given that the interface unit is a connected device that has a powerful processor (or processors) and a number of familiar I/O components including, for example, a touch sensitive display screen, Wi-Fi, cellular and other wireless communications capabilities, a speaker, a microphone and optionally a camera, the interface unit can be programmed to provide a number of useful functionalities without impacting the functionality of the base defibrillator unit in any way. Thus, it should be apparent that the described modular architecture helps overcome a number of practical challenges that have hindered widespread adoption of defibrillator connectivity.

Although only a few embodiments of the invention have been described in detail, it should be appreciated that the invention may be implemented in many other forms without departing from the spirit or scope of the invention. The description sometimes refers to independent monitors. For context, there are a number of advanced cardiac life support (ACLS) systems that combine defibrillation functionality (often in both manual and automated modes) with functionalities such as cardiac monitoring, pacing, pulse oximetry, capnography, blood pressure monitoring, etc.. However, the monitors used in such systems are not actually independent systems. Rather, they are all part of the same life support system. In contrast, in some of the described embodiments, the monitor has its own ecosystem and data reporting pathways and it is not reporting its data directly to the AED, a life support system that includes the defibrillator, or an AED management server.

In many of the examples above, the ability to present independent monitor data as part of an AED incident report in real time during an incident and/or very quickly after the deployment of an AED has been terminated is emphasized. While that is very valuable from a medical perspective, the ability to integrate the reports can be useful even when there is an extended delay in receiving the monitor data. This is because the integrated report is a useful tool for cardiologists and others seeing the patient well after the incident. Thus, the described system is very useful even in conjunctions with monitor systems that are not able to provide their patient data in near real-time or shortly after an incident.

The discussion above refers to several different messages that are sent between various nodes in the system. It should be appreciated that the described messages may take the form of a single or multiple separate communications and/or packets.

In certain embodiments described herein, a pull-type data retrieval model is employed for obtaining monitor data from the monitor management server. In such models, the incident report server actively requests monitor data as needed. However, in alternative embodiments, a push-type data delivery model or a hybrid model may be preferable. For instance, when the monitor management server is aware of, or is informed of, an incident identifier, it may proactively determine which monitor data is pertinent to that incident and transmit such data to the incident report server without awaiting a request. Indeed, if desired, push models can be used any time the monitor management server has (and conveys) enough information about the incident (e.g., time/location) for the incident report server to be able to associate the received monitor data with the corresponding incident.

In some of the embodiments described above, the incident reports are provided in a format that is well suited for review by medical personnel. However, in other systems, a patient record system may be interested in some of the underlying data for presentation using its own user interface and the incident report server can readily provide any desired underlying data either alone, or in combination with a more human readable incident report.

The described systems can be used in conjunction with a variety of different defibrillators. However, they are particularly compelling in the context of AEDs, because AEDs are typically designed for use by most anyone rather than being limited to use by medical professionals and/or in formal medical settings and/or as part of an advanced cardiac life support (ACLS) device/system. At the same time, there are a number of medical devices (including prescribed cardiac monitors and other medical devices), as well as a proliferation of consumer products that may be monitoring a victim that suffers a cardiac arrest – that may have useful information about the incident, but today that information rarely gets to the emergency medical personnel that initially treat the patient. Thus, the described system offers the possibility of getting useful event information into the hands of medical professionals for use in making diagnostic and treatment decisions for cardiac arrest victims in circumstances where such information simply isn’t available today.

In the descriptions above, specific power control electronics and control processes for managing power supplied by a supplemental power bank are described. However, it should be apparent that the described functionalities may be implemented using a variety of different control electronics or may be implemented in large part via one or more programmed processors that execute programmed instructions stored in memory associated with the processor(s).

Several flow charts have been provided to help describe some of the major functions provided by the systems. It should be appreciated that to the extent that performance isn’t compromised, the order of some of the steps may be rearranged and/or some of the steps may be performed substantially concurrently. Similarly, some described steps can be combined or eliminated, while others may be added. Therefore, the present embodiments should be considered illustrative and not restrictive, and the invention is not to be limited to the details given herein, but may be modified within the scope and equivalents of the appended claims.

What is claimed is:

1. A method comprising:
by a server infrastructure, receiving incident data from a first automated external defibrillator (AED) regarding a use of the first AED in connection with a medical incident;
by the server infrastructure, receiving or retrieving a monitor identifier that identifies a first monitor that is independent of the first AED that at least one of (i) is/was in a vicinity of the medical incident during the use of the first AED, (ii) was pre-associated with the first AED prior to the medical incident, (iii) is/was able to detect the first AED during use of the first AED, or (iv) was associated by identifying a patient following the use of the first AED;
requesting monitor data from a monitor management server associated with the first monitor using the monitor identifier;
by the server infrastructure, receiving the requested monitor data; and
by the server infrastructure, generating an incident report that combines at least some of the received AED incident data and at least some of the received monitor data.

2. A method as recited in claim 1 wherein the first monitor was pre-associated with the first AED prior to the medical incident and the monitor identifier is retrieved from an AED management database that stores information about a multiplicity of AEDs, including the first AED.

3. A method as recited in claim 1 wherein the monitor identifier is received from the first AED.

4. A method as recited in claim 3 wherein during the use of the first AED in connection with the medical incident, the first AED scans for nearby monitors and the monitor identifier was obtained by the first AED as part of the scan for nearby monitors.

5. A method as recited in claim 4 wherein the scan is performed using a near field scan selected from the group consisting of:
an RFID scan;
a Bluetooth scan;
a Bluetooth Low Energy scan;
a Wi-Fi scan;
a Near-Field Communication scan;
a Dedicated Short-Range Communications scan; or
a Zigbee or Z-Wave scan.

6. A method as recited in claim 3 wherein the monitor identifier was input into the first AED after the use of the first AED in connection with the medical incident.

7. A method as recited in claim 1 wherein the monitor identifier is received from the monitor management server during or after the use of the first AED in connection with the medical incident.

8. A method as recited in claim 1 wherein the identification of the patient following the use of the first AED is used, at least in part, to obtain the monitor identifier.

9. A method as recited in claim 1 further comprising:
creating an incident record in response to reception of incident data from the first AED, wherein the incident report is generated based at least in part on information in the incident record; and
making the incident report available to a requestor.

10. A method as recited in claim 1 further comprising, making the incident report available to a requestor during at least one of: (i) the use of the first AED; and (ii) the medical incident.

11. A first automated external defibrillator (AED) comprising (i) a shock discharge capacitor, (ii) at least one defibrillation electrode pad, the shock discharge capacitor and the at least one defibrillation electrode pad being suitable for delivering a defibrillation shock to a potential cardiac arrest incident victim during a first emergency use of the first AED, and (iii) one or more processors collectively configured to:
in association with the first emergency use of the first AED, electronically scan for nearby monitor devices;
cause a detected device list to be electronically transmitted directly or indirectly to a remotely located server infrastructure, wherein the detected device list identifies at least one device detected by the electronic scan;
in association with the first emergency use of the first AED, generate a first incident log that contains event information related to the first emergency use of the first AED; and
cause the first incident log to be electronically transmitted directly or indirectly to the remotely located server infrastructure, wherein the first incident log includes at least one of a first incident identifier, and elements that can be used to generate the first incident identifier and wherein the server infrastructure is configured to receive incident logs from a multiplicity of different AEDs, including the first AED.

12. The first AED as recited in claim 11 further comprising an RFID reader that, at least in part, performs the scan for nearby monitor devices.

13. The first AED as recited in claim 11 further comprising a BLE communications module that, at least in part, performs the scan for nearby monitor devices.

14. The first AED as recited in claim 11 further comprising a scanner that performs the scan, the scanner including or being embodied in at least one selected from the group consisting of:
a Bluetooth communications module;
a Wi-Fi communications module;
a Near Field communications module;
a Dedicated Short-Range communications module;
a Zigbee communications module; or
a Z-Wave communications module.

15. The first AED as recited in claim 11 further comprising a display screen, and wherein the one or more processors is/are further configured to cause the first incident identifier to be rendered on the display screen after the first emergency use of the first AED.

16. The first AED as recited in claim 11 wherein the one or more processors is/are further configured to cause status messages to be periodically sent to the server infrastructure during the first emergency use of the first AED.

17. A first automated external defibrillator (AED) comprising (i) a shock discharge capacitor, (ii) at least one defibrillation electrode pad, the shock discharge capacitor and the at least one defibrillation electrode pad being suitable for delivering a defibrillation shock to a potential cardiac arrest incident victim during a first emergency use of the first AED, (iii) a display screen, and (iv) one or more processors collectively configured to:
in association with the first emergency use of the first AED, generate a first incident log that contains event information related to the first emergency use of the first AED;
cause the first incident log to be electronically transmitted directly or indirectly to a remotely located server infrastructure, wherein the first incident log includes at least one of a first incident identifier, and elements that can be used to generate the first incident identifier and wherein the server infrastructure is configured to receive incident logs from a multiplicity of different AEDs, including the first AED;
receive a user input that provides at least one of (a) a monitor identifier that identifies a monitor associated with a patient that the first AED was used on during the first emergency use of the first AED, or (b) a patient identifier that identifies the patient; and
forward the received monitor identifier or patient identifier to the server infrastructure.

18. A patient monitoring system comprising:
a monitor server infrastructure;
a first monitor located remotely relative to the monitor server infrastructure, the first monitor configured to (i) monitor a physiological characteristic of a patient, (ii) send monitor data regarding the monitored patient’s physiological characteristic to the monitor server infrastructure, (iii) detect a deployment of a first defibrillator, and (iv) notify the monitor server infrastructure of the detection of the deployment of the first defibrillator; and
wherein the monitor server infrastructure forwards at least some of the received monitor data to a remotely located incident management server infrastructure to facilitate inclusion of at least some of the forwarded monitor data in an incident report that combines incident data collected by the first defibrillator during the deployment of the first defibrillator.

19. A patient monitoring system as recited in claim 18 wherein:
in response to being notified of the detection of the deployment of the defibrillator, the monitor server infrastructure sends an incident query to the incident management server infrastructure; and
at least some of the forwarded monitor data is sent to the incident management server infrastructure in response to a monitor data request sent by the incident management server infrastructure to the monitor server infrastructure.

20. A patient monitoring system as recited in claim 18 wherein the first defibrillator is an automated external defibrillator (AED).
